(19) United States
(12) Patent Application Publication
(10) Pub. No.: US 2002/0128710 A1
(43) Pub. Date: Sep. 12, 2002

(54) Modular intraocular implant

(76) Inventor: Harry C. Eggleston, Creve Coeur, MO (US)

Correspondence Address: Paul M. Denk, 763 South New Ballas Road, St. Louis, MO, 63141, US

(21) Appl. No.: 10/142,486

(22) Filed: May 10, 2002

Related U.S. Application Data
(63) Continuation-in-part of application No. 09/372,493, filed on Aug. 20, 1999.
Application No. 09/372,493 is a continuation-in-part of application No. 08/764,501, filed on Dec. 12, 1996, now abandoned.
Application No. 08/764,501 is a continuation-in-part of application No. 08/617,183, filed on Mar. 18, 1996, now Pat. No. 5,628,798.

(51) Int. Cl. A61F 2/16

(52) U.S. Cl. 623/6.22; 623/6.41

(57) ABSTRACT

An adjustable ocular insert to be implanted during refractive cataract surgery and clear (human) crystalline lens refractive surgery and adjusted post-surgically. The implant comprises relatively soft but compressible and resilient base annulus designed to fit in the lens capsule and keep the lens capsule open. Alternatively the annulus may be placed in the anterior or posterior chamber. The annulus can include a pair of opposed haptics for secure positioning within the appropriate chamber. A rotatable annular lens member having external threads is threadedly engaged in the annulus. The lens member is rotated to move the lens forward or backward so to adjust and fine-tune the refractive power and focusing for hyperopia, myopia and astigmatism. The intraocular implant has a power range of approximately +3√0Π−3 diopters.

### CROSS REFERENCE TO RELATED APPLICATIONS

This application is a continuation in part of application Ser. No. 09/372,493, filed Aug. 20, 1999, which is a continuation in part of application Ser. No. 08/764,501, filed Dec. 12, 1996, which is a continuation in part of application Ser. No. 08/617,183, filed Mar. 18, 1996, now U.S. Pat. No. 5,628,798.

### BACKGROUND OF THE INVENTION

This invention relates generally to ocular implants and more specifically to a modular intraocular implant with an adjustable and replaceable lens.

A cataract is a condition where a normally clear lens of the eye becomes progressively opaque. The opacification generally occurs over a period of time and the amount of light which passes through the lens decreases thereby decreasing vision. It is necessary, therefore, to surgically remove and replace the clouded lens. Often, there is a coexistent refractive defect such as myopia (short sightedness), hyperopia and astigmatism.

Generally the lens is removed for cataract or clear lens refractive purposes for high myopia and hyperopia, and is replaced at the time of surgery with an intraocular lens formed from a biocompatible material such as PMMA (polymethyl methacrylate) or the like. The surgeon makes an incision in the sclera and cornea to allow the removal of the semi-opaque lens and/or clear lensectomy and insertion of the implant. The typical prior art lens implant is either of plano-convex design or double convex design, with each curved surface defining a spherical section. A large number of patients will have significant post-surgical astigmatism and spherical error and will need a spherical/astigmatic adjustment in their glasses (or an operative corneal astigmatic relaxing incision).

The surgery and healing may induce myopia and/or hyperopia with biological healing (e.g. aging) and there can be fluctuations greatly over time following the surgery as the capsular bag, lens zonules, cornea, etc. change.

One problem associated with intraocular lens implants is that it is necessary to decide, preoperatively, on the power of the lens. This can be done by performing various standard clinical ultrasound and optical laser-like measurements of the anterior posterior corneal distance, anterior chamber depth, etc. and then making an estimate of the proper power of the IOL to determine the proper post-operative refraction of the eye. Although the ophthalmologist (eye M.D.) uses the best techniques available, it is very difficult to accurately predict, preoperatively, the optimal power for the lens implant because of multiple variables of axial length, anterior chamber depth, corneal curvature and size, growth of the eye (pediatric cases), irregular post scleral surfaces (usually seen in the macular area) such as myopic staphylomas, mismeasurement and mislabeling of the IOL power and other human errors. Therefore, most patients are required to use glasses for precise focusing even after the replacement of the semi-opaque lens. Further, since the exact amount and axis of astigmatism cannot be accurately determined until several weeks after surgery, the patient may require glasses for best vision and the lens prescription may have to be changed more than once as the eye heals over time, with normal physiological aging and because of different visual needs.

Several intraocular lenses which allow post-surgical correction are known. U.S. Pat. No. 4,575,373 discloses a laser adjustable intraocular lens. U.S. Pat. No. 4,816,031 provides a lens implant with a second soft and pliable lens position over it and electromechanical circuitry for regulating the distance between the two lenses.

U.S. Pat. No. 4,601,545 discloses a variable power lens having optically active molecular material, such as liquid crystals than can be configured using electrical voltages. U.S. Pat. No. 4,564,267 discloses a variable focal length lens which can be electrically controlled by applying an electric field to a compound lens with one lens formed of electrooptic crystals.

U.S. Pat. No. 4,373,218 discloses a variable power intraocular lens including a fluid expandable sac for containing a liquid crystal material that responds to electric charge to change the index of refraction of the lens.

U.S. Pat. No. 4,932,966 discloses a intraocular lens apparatus having a flexible lens member and with a relatively rigid portion with fluid-filled chambers therebetween. The shape or position of the lens portion is adjusted by changing fluid pressure in the fluid-filled chambers.

U.S. Pat. No. 4,932,971 provides a clip-on optic assembly for clipping in situ onto a previously implanted intraocular lens to change its optical characteristics without removal from the eye.

U.S. Pat. No. 5,108,429 provides an adjustable focus lens with a plurality of micromotor devices spaced around the periphery of the lens body, the devices being responsive to an external control signal for selectively changing the position of a lens body. U.S. Pat. No. 5,203,788 also provides an adjustable lens apparatus having a lens body with a relatively rigid outer ring with micromotors between the lens body and the outer ring that are responsive to outside actuation.

U.S. Pat. No. 5,171,266 discloses an intraocular lens having a flexible lens body center portion surrounded by an outer ring which is sensitive to an external force such as a magnetic force. The shape of the outer body can be changed by magnetic force to elongate the lens body. U.S. Pat. No. 5,326,347 also discloses an intraocular implant that is responsive to the post-surgical application of force, such as the movement of the implantee's head and magnetic force to change the focus.

Although the foregoing devices may solve the problem of adjustment of the lens post-surgically, there have other inherent drawbacks. Some of the adjustable lenses are complicated in design employing power sources, micromotors, microfluid pumps and electric or electrochemical circuitry. Such complex devices can be expensive to manufacture and relatively bulky or heavy in use. Some adjustable lenses require the use of external adjustment technology such as electric current, magnets or other forces.

### SUMMARY OF THE INVENTION

It is, therefore, among the principal objects of the present invention to provide an adjustable ocular implant, even one that can be adjusted in situ.

It is another object of the present invention to provide an adjustable and upgradeable intraocular lens implant to allow for improvements in optical resolution, wave length management and new technologies that can be adjusted post-surgically to improve focus, resolution quality and the filtering out of harmful light, or even accidental laser rays.

It is yet another object of the present invention to provide an adjustable intraocular implant that is relatively simple and elegant in design.

Still another object of the present invention is to provide an adjustable intraocular implant that is lightweight and easily implanted during cataract surgery.

Yet another object of the present invention is to provide an adjustable intraocular implant that allows for the simple replacement of a corrective lens.

Another object of the present invention is to provide an adjustable intraocular implant that does not require the use of complex techniques or peripheral devices to effect adjustment of the implant after surgery.

Yet another object is to allow with the same general machining and overall structural design for different plastic and glass-like materials of rigid and soft compressible natures for small surgical incisions and different economic budgets.

Yet another object of the invention is to provide an adjustable intraocular implant that will adjust and fine-tune the refractive power of the lens of the eye and provide for focusing for hyperopia, myopia, astigmatism and accommodation with advanced magnetic model.

In accordance with the invention, an adjustable intraocular implant is provided that can be implanted during cataract surgery and/or clear lensectomy and easily adjusted post-surgically. The implant comprises relatively soft but compressible and resilient outer or base annulus designed to fit in the lens capsule and keep the lens capsule open. Alternatively the annulus may be placed in the anterior or posterior chamber. There is a second concentric annulus removably seated in the outer annulus. The second annulus can have a threaded inner surface or be lined with a threaded insert.

A rotatable annular lens mount, bearing an appropriate lens, is threadedly engaged in the second annulus. The lens mount can be rotated with an appropriate tool to move the lens forward or backward so to adjust and fine-tune the refractive power and focusing for hyperopia, myopia and astigmatism. The intraocular implant has a correction range for spherical correction of approximately +3√0Π−3 diopters with probable steps of 0.50 diopters and for astigmatic correction of +1 to +4 with an overlay lens. The second annulus can be removed from the base annulus and lifted out of the eye so that the rotatable lens assembly can be changed with less stress on the base annulus lens zonule capsule on the rotatable lens assembly. An alternative embodiment of the intraocular lens includes a flexible, base annulus. The base annulus defines a central, internally threaded circular opening The base annulus includes two opposed curvilinear haptics extending from the outer edge for securing the intraocular implant in the posterior capsular bag and/or cilary sulcus. The intraocular lens includes a lens member having a threaded outer edge rotatably mounted within the base annulus. The lens member can be rotated within the base annulus for adjustment.

In another embodiment of the invention, the lens member is rotatably mounted within the base annulus. The lens includes magnetic material adjacent the periphery of the lens member. The lens can be rotated in situ by manipulating a magnetically attractive tool, such as an electromagnetic device that is capable of fine-tuning or computer operation.

### BRIEF DESCRIPTION OF THE DRAWINGS

FIG. 1 is an isometric view of the adjustable ocular implant of the present invention;

FIG. 2 is a front plan thereof;

FIG. 3 is a cross-sectional view thereof taken along line 3-3 of FIG. 2;

FIG. 4A is a front plan of the clear lens of the adjustable ocular implant of the present invention;

FIG. 4B is a side elevational view thereof;

FIG. 5A is a front plan of one preferred embodiment of the lens mount of the adjustable ocular implant of the present invention;

FIG. 5B is a slide elevational view thereof;

FIG. 5C is a side elevational view of another embodiment of the lens mount of the adjustable ocular implant of the present invention;

FIG. 5D is a side elevational view of another embodiment of the lens mount of the adjustable ocular implant of the present invention;

FIG. 6A is a top plan of a threaded insert of the adjustable ocular implant of the present invention;

FIG. 6B is a side elevational view thereof;

FIG. 7A is a top plan of the second annulus of the adjustable ocular implant of the present invention;

FIG. 7B is a side elevational view thereof;

FIG. 7C is a bottom plan thereof;

FIG. 7D is another side elevation view thereof;

FIG. 8A is a front plan of the base annulus of the adjustable ocular implant of the present invention;

FIG. 8B is a side elevational view thereof;

FIG. 8C is a partial section of the base annulus of FIG. 8A;

FIG. 8D is a cross-sectional view taken along line 8D-8D of FIG. 8C;

FIG. 9A is another front plan of the adjustable ocular implant of the present invention;

FIG. 9B is another cross-sectional view of the adjustable ocular implant of the present invention taken along line 9B-9B of FIG. 9A;

FIG. 10A is a front plan of one preferred embodiment of the lens mount of the adjustable ocular implant of the present invention;

FIG. 10B is a side elevational view thereof;

FIG. 11A is a front plan of another preferred embodiment of an adjustable ocular implant of the present invention;

FIG. 11B is a cross-sectional view of the adjustable ocular implant of the present invention take along line A-A of FIG. 11B;

FIG. 12 is an exploded view of another embodiment of the adjustable ocular implant of the present invention;

FIG. 12A is an exploded view of yet another embodiment of the adjustable ocular implant of the present invention;

FIG. 13 is a front plan of another preferred embodiment of an adjustable ocular implant of the present invention;

FIG. 14 is a side elevational view thereof;

FIG. 15 is a front plan of the outer ring for use with the adjustable ocular implant of FIGS. 14 and 15;

FIG. 16 is a side elevational view thereof;

FIG. 17 is a front plan of another preferred embodiment of an adjustable ocular implant of the present invention;

FIG. 18A is a front plan of one embodiment of the lens of the adjustable ocular implant the adjustable ocular implant of FIG. 17;

FIG. 18B is a front plan of another embodiment of the lens of the adjustable ocular implant the adjustable ocular implant of FIG. 17; and

FIG. 19 is a side elevation view of the lens of FIGS. 18A and 18B.

FIG. 20 is a perspective view of a lens button component of a magnetic lens blank of the present invention;

FIG. 21 is a magnetic ring;

FIG. 22 is a perspective view of the lens cap component of the magnetic lens blank;

FIG. 23 is an exploded view of the magnetic lens blank before assembly;

FIG. 24 is a perspective view of the assembly magnetic lens blank before threading and machining, and

FIG. 25 is a perspective view of the assembly lens.

Corresponding reference figures indicate corresponding structures throughout the various drawings.

### DESCRIPTION OF THE PREFERRED EMBODIMENT

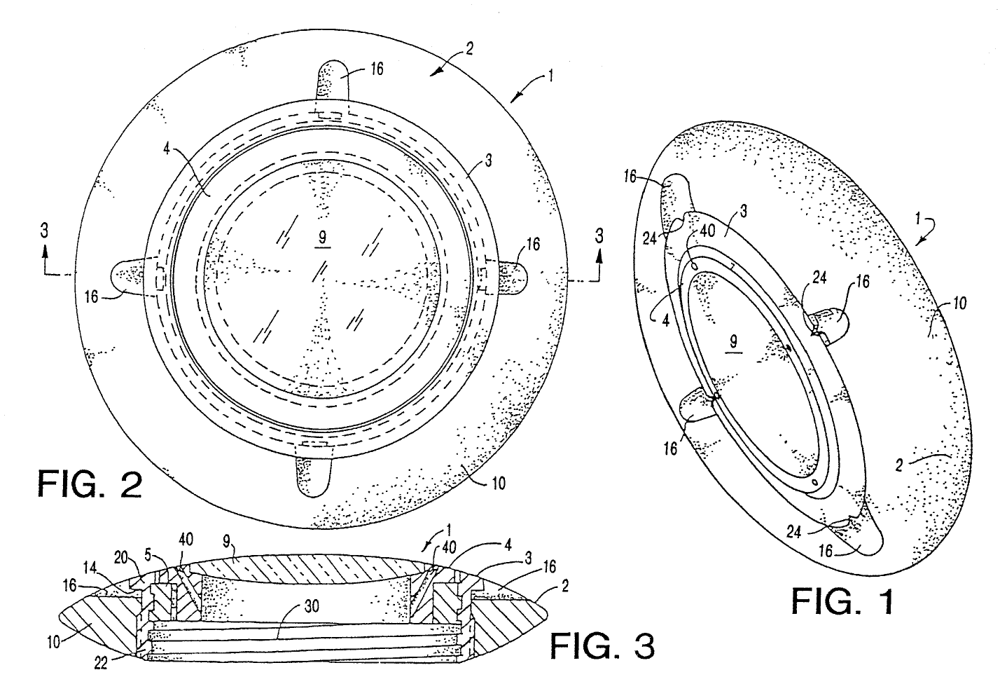

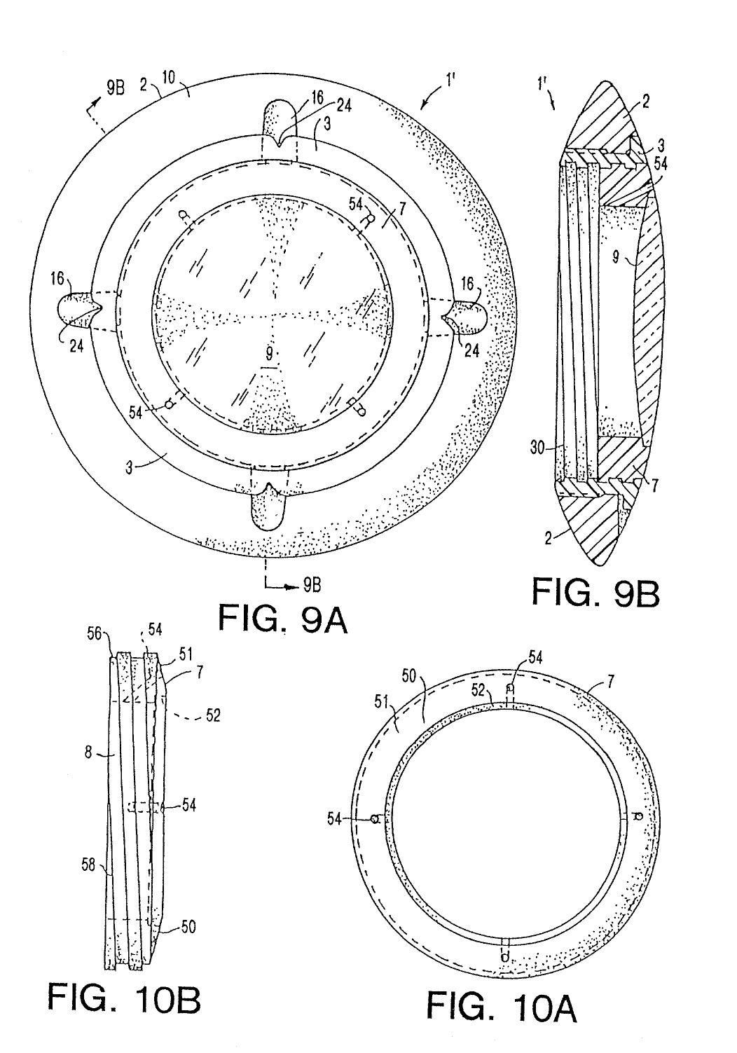

The adjustable intraocular insert of the present invention is indicated generally by reference numeral 1 in the drawings. Insert 1 has several principal components including an outer or base annulus 2 and a concentric second annulus 3. An adjustable lens mount assembly, indicated generally by reference numeral 4 in FIGS. 5A and 5B, is rotatably mounted in second annulus 3. In the preferred embodiment of FIGS. 1-3, the lens mount assembly 4 comprises two pieces, employing an externally threaded ring 5. Another embodiment of the adjustable intraocular insert, indicated generally be reference numeral 1′ in FIGS. 9A and 9B, employs a one-piece lens holder 7 (FIGS. 10A and 10B) which includes an externally threaded annular skirt 8. In any event, the lens holder seats a refractive lens 9. The various components of the adjustable intraocular insert will now be described in greater detail.

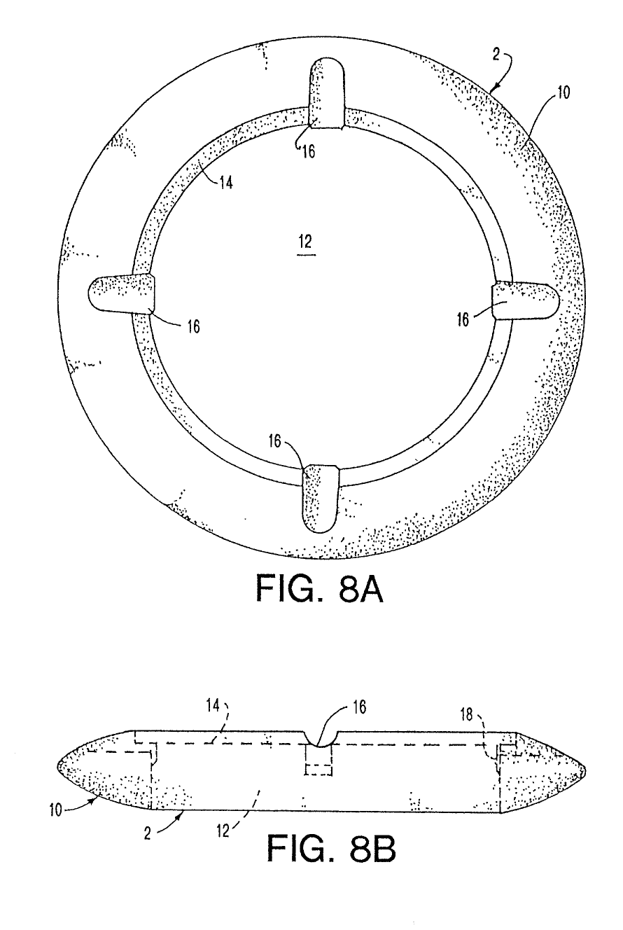

The base annulus 2, shown in greater detail in FIGS. 8A-8D, is sized and configured to fit into the capsular bag of the human eye after a cataract has been removed. Alternatively, annulus 2 can be configured to be placed in the ciliary sulcus or anterior chamber. Annulus 2 has a compressible, resilient annular body 10 made of a biologically compatible, relatively inert material such as PMMA, silicone or the like. As shown, body 10 defines a central, substantially circular opening 12. There is a rabbet 14 formed around the peripheral edge of opening 12 to seat the second annulus 3, as will be described below. Also formed in the face of body 19 is a plurality of indentions or notches 16. In the illustrated embodiment, four notches 16 are formed in the face of body 10 and spaced equidistant around the face of the body. The notches 16 are in communication with rabbet 14 and are formed to allow an ophthalmologist or technician access to the second annulus as will be explained below. As can best be seen in FIGS. 8C and 8D, there is a raised detent 18 formed on the inner surface of the annulus. There is a second detent 18 directly across from the first. Detents 18 are designed to engage complementary slots formed in the second annulus 3 in a bayonet-type lock, as will now be explained.

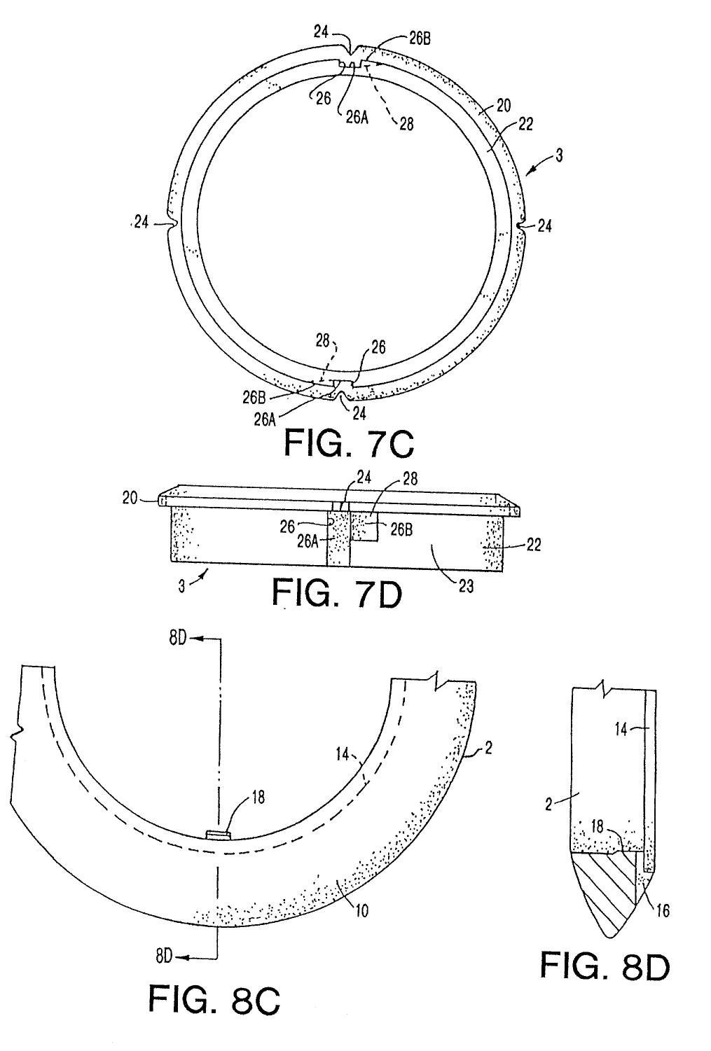

The second annulus 3 is best shown in FIGS. 7A and 7B. Annulus 3 has outer ring 20 with a beveled leading edge 21 and a concentric annular skirt 22. Skirt 22 has a generally smooth outer surface 23. It will be appreciated that outer ring 20 is dimensioned to seat in rabbet 14 of the base annulus when the intraocular insert is fully assembled. Outer ring 20 has a plurality of V-shaped grooves 24 formed therein. The embodiment shown includes four grooves 24 placed equidistant around the ring which are designed to accept a tool for the application of torque so as to rotate annulus 3 for the introduction or removal of annulus 3 from base annulus 2. It will be appreciated, therefore, that grooves 24 must align with notches 16 in the base annulus when second annulus 3 is appropriately positioned and locked therein.

As shown in FIG. 7D, a substantially L-shaped locking channel 26 is formed in opposite sides of the external surface 23 of skirt 22. Each locking channel 26 has two sections, a substantially rectangular section 26A and a substantially square section 26B. As seen in FIG. 7C, section 26A has a uniform depth. However, section 26B has a bottom ramp 28. That is, the depth of section 26B decreases from the juncture with section 26A to the juncture with the outer surface 23 of skirt 22. Thus, ramp 28 angles up from section 26A to surface 23.

As should be appreciated, the L-shaped locking groove 26 is intended to align with the detents 18 of annulus 2. When skirt 22 of second annulus 3 is inserted into opening 12, the detents 18 engage section 26A of locking groove 26. Annulus 3 is urged into the base annulus until outer ring 20 is seated in rabbet 14. Once properly seated, an appropriate tool is inserted through notches 16 to engage V-shaped grooves 24 on the outer ring of the second annulus. Annulus 3 is rotated so that the detents 18 slide up ramps 28 of groove section 26B. Annulus 3 thus is held in place by the bayonet-like lock. Moreover, there is a snug friction fit between the respective detents 18 and the ramps 28. The second annulus can be removed from the base annulus and lifted out of the eye so that the rotatable lens assembly can be changed with less stress on the base annulus and on the rotatable lens assembly. As seen in FIGS. 3 and 7B, the inside of skirt 22 has threads 30. Threads 30 are designed to threadedly engage a lens mount assembly.

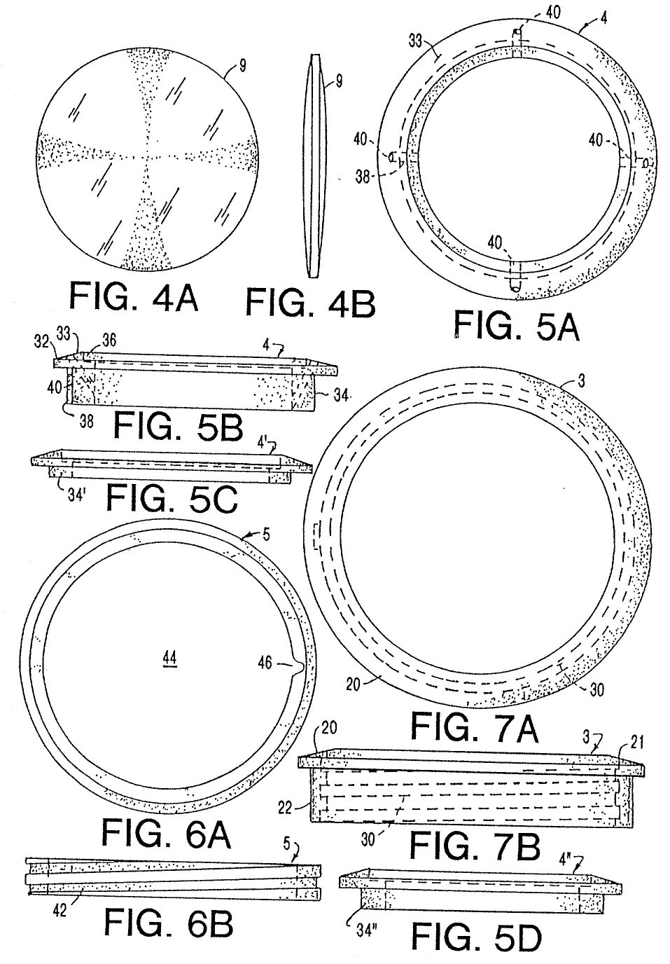

In one preferred embodiment, as stated above, the lens mount assembly consists of a lens mount 4, shown in FIGS. 5A and 5B and threaded ring 5, shown in FIGS. 6A and 6B. As best seen in FIG. 5A, lens mount 4 has an outer ring 32 with beveled face 33 and a concentric annular skirt 34. Ring 32 has a rabbet 36 dimensioned to seat a bi-convex lens 9 (FIGS. 4A and 4B) or other appropriate refractive lens. Lens mount 4 can hold a filter or other element, as will be discussed below. Also, an overlying soft pliable toric lens of silicone, Teflon or other biocompatible material can be placed over the top of lens 9 if required, rather than in the lens chamber with separate slip friction ring for spherical power or astigmatic correction. Skirt 34 has an alignment spine 38 on the outer surface. A plurality of fluid flow channels 40 extends from face 33 through ring 32 and through the skirt. The fluid channels 40 are formed at an angle of approximately 60° relative to the axis of the lens mount and function to allow the ingress and egress of ocular fluids and maintain and even fluid pressure on both sides of the insert. In another embodiment, the fluid channels could be filled or impregnated with pharmaceutical agents such as anti-inflammatory drugs, antibiotics or glaucoma agents for treatment of particular diseases. Alternatively, small pockets (not shown) filled with drugs could be included in the lens holder (or the base annulus or second annulus). It may possibly be important to point out that different laser energies may be used to allow for the slow release of this medication, such as photodisruptive lasers (YAG'S) or thermolasers (argon or krypton). The pockets could be opened with laser energy to allow the slow release of the medication.

The lens mount 4 is designed to seat in threaded ring 5, as shown in FIG. 6B. Ring 5 has a smooth interior surface and external threads 42. Threads 42 are disposed to engage the internal threads 30 of skirt 22 of second annulus 3 as will be explained below. Threaded right 5 defines and generally circular opening 44 dimensioned to seat skirt 34 of lens mount 4 with a snug friction fit. There is an alignment groove 46 formed in the internal face of the ring. During assembly, alignment spline 38 of lens mount 4 is positioned in groove 46 to ensure appropriate positioning. Once lens mount 4 is snugly secured in ring 5, ring 5 can be threadedly engaged in the second annulus. As will be appreciated, the entire lens mount assembly can be rotated within the second annulus to focus lens 9.

The multiple piece lens mount assembly just described affords added flexibility in that ring 5 can remain threadedly engaged in annulus 3 when lens mount 4 is extracted from its snug friction fit within ring 5 to change lens 9, for example. This arrangement allows for less trauma to other components of the insert and to the patient.

Alternatively, adjustable ocular insert 1′ illustrated in FIGS. 9A and 9B employs a lens mount of a unitary construction, indicated by reference numeral 7. Lens mount 7, best seen in FIGS. 10A and 10B, has an outer ring 50 having a beveled face 51 defining a rabbet 52 to seat a lens 9 or the like. Lens mount 7 also has a plurality of fluid paths 54 of the design previously described. Lens mount 7, however, has an integral, externally threaded skirt 56 with threads 58 designed to engage the internal threads of second annulus 3. Lens mount 7 is simpler and employs one piece. However, it does not afford the flexibility of the previously described lens mount assembly. That is, the entire lens mount 7 must be unscrewed from annulus 3 in the event lens 9 must be changed.

Regardless of the design of the lens mount, the lens mount can be rotated within annulus 2 to change the focus of the lens. The lens mount is rotated to move the lens forward or backward so to adjust and fine-tune the refractive power and focusing for hyperopia, myopia astigmatism and accommodation. The intraocular implant has a power range of approximately 1 to 3 diopters + and −. For example, predetermined movement of the lens mount could result in a predetermined change in power. For example, rotation of the lens holder 1 mm could result in a change of 3 diopters; 0.5 mm of rotation inside the eye could have plus or minus 1.5 diopters of focal power in the back of the eye. Therefore, the device will have corrections range of 0 to +3 diopters and 0 to −3 diopters. Prism range is from 2 to 6 with the prism base up, down, in, out or obliquely as desired. The prism will be rotary and can be turned to any meridian, other than the four cardinal directions.

It should be noted that fixed marking could be made on the face of the lens holder 4 and the second annulus 3 or base annulus 2 so that alignment of the respective marks would result the setting of a predetermined dioptric power. That would make precise adjustment predictable.

Various changes and modifications may be made in the ocular implant of the present invention without departing from the scope of the appended claims. For example, FIG. 5C illustrates another lens holder, indicated generally as 4′. It will be noted that lens holder 4′ has a substantially shorter skirt 34′. Another lens mount, indicated generally by reference numeral 4″ in FIG. 5D has an intermediately sized skirt 34″. By varying the length of the respective skirts 4′, 4 and 4″, the physician can vary the surface area of contact between the lens holder and ring 5. If it is anticipated that the lens will be changed frequently, lens holder 4′ or 4″ may be employed since they would be easier to withdraw from ring 5. On the other hand, lens holder 4, although still removable, is more difficult to extract than other embodiments.

Since the lens holder is removable, the ocular insert of the present invention allows for upgradability of ocular or lens material to allow for greater optical resolution and purity. Further, various lenses, prisms, filters such as U.V., polarizing, infrared, blue light or photochromic filters and/or lenses, and/or the lens holder could carry combinations or permutations. Moreover, base annulus 2 can serve as a mounting means for future innovation in optics such as electro-optical devices, photosensors, photo power packs and mechanical medical devices. Sighting or alignment devices also could be employed. For example, lighting lines and leveling lines can be adjusted for equilibrium, orientation, measurement and stability. Also, it will be appreciated that an overlay lens can be placed over the lens for astigmatic correction of +1 to +4.

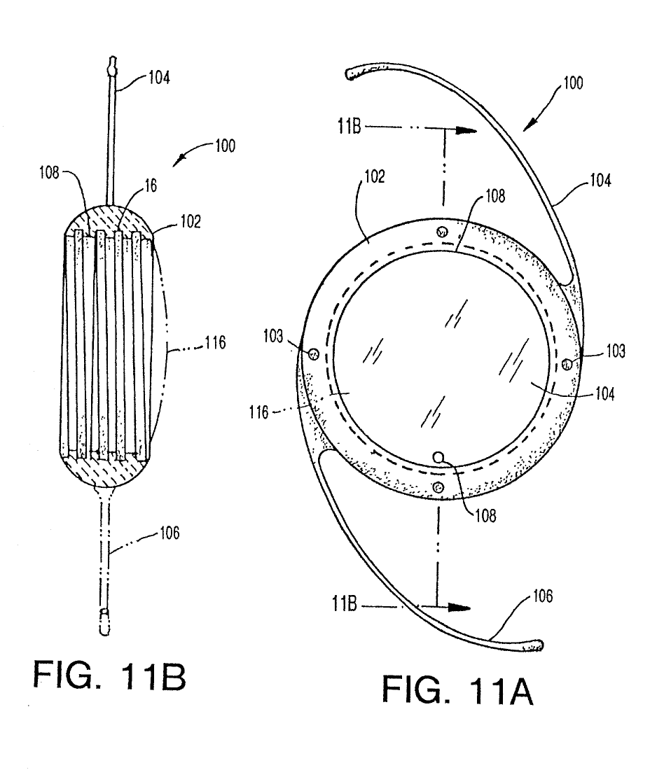

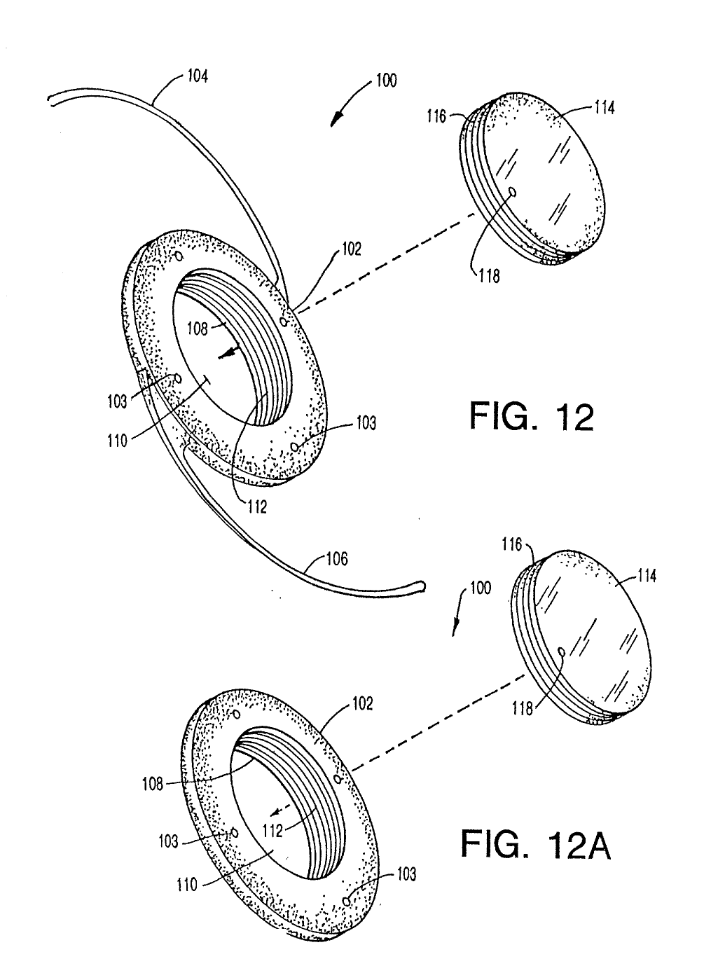

Another embodiment of the adjustable ocular implant is illustrated in FIGS. 11A through 12A, and is indicated generally by reference numeral 100. Implant 100 includes a base annulus 102. Base annulus 102 can be constructed from same or similar materials as previously described relative to base annulus 2. Preferably the material is resilient and somewhat flexible for introduction into the eye through an incision. Annulus 102 includes a plurality of openings 103 formed in the face to allow the introduction of an instrument for proper positioning when surgically inserted. The openings 103 also serve as indicia of dioptric correction, as will be explained in greater detail below.

Annulus 102 of FIG. 12 has a first curvilinear haptic 104 extending out from the outer edge and a second curvilinear haptic 106 on the outer edge opposite the first haptic. The haptics 104,106 allow the secure positioning of the ocular insert in the posterior capsular bag or the anterior chamber or the like. The base annulus 102 includes an inner wall 108 which defines a central circular opening 110. Wall 108 includes grooves 112 for the threaded engagement of a lens as will now be described. FIG. 12A illustrates a similar embodiment without the haptics. Intraocular implant 100 includes a lens member 114 which is dimensioned to seat in opening 110. Lens member 114 is bi-convex constructed from appropriate lens material. Lens 104 includes a threaded outer peripheral edge 116 disposed to engage the grooves 112 in the interior surface of the base annulus. Lens member 114 includes at least one opening 118 in the face for the insertion of an instrument to allow rotation of the lens member within the base annulus which moves the lens member forward or backward within the base annulus. Further, alignment of the opening 108 with a specific opening 103 of the base annulus results in a predetermined dioptric power. Thus, the surgeon can adjust the corrective power of the intraocular implant by the appropriate rotation of the lens member within the base annulus. The intraocular implant has a power range of approximately +3√0Π−3 diopters. Further, lens member 114 can be rotated until it is freed from the base annulus and replaced with a lens member having a different dioptric power.

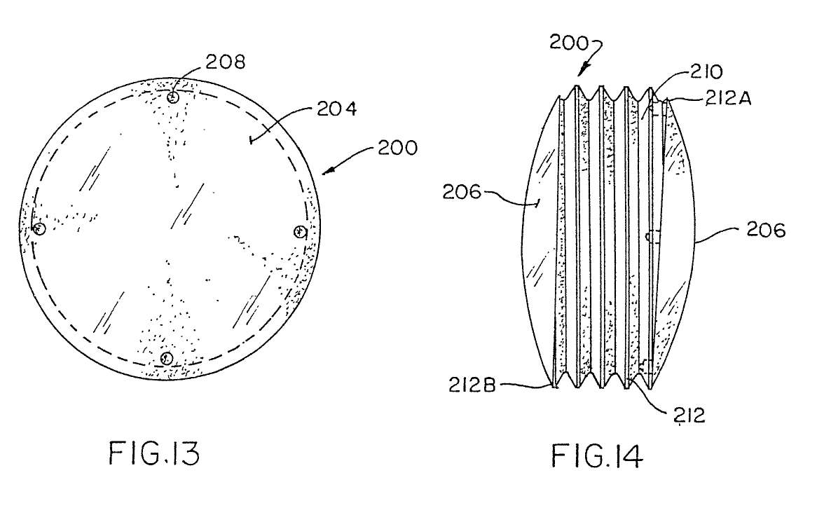

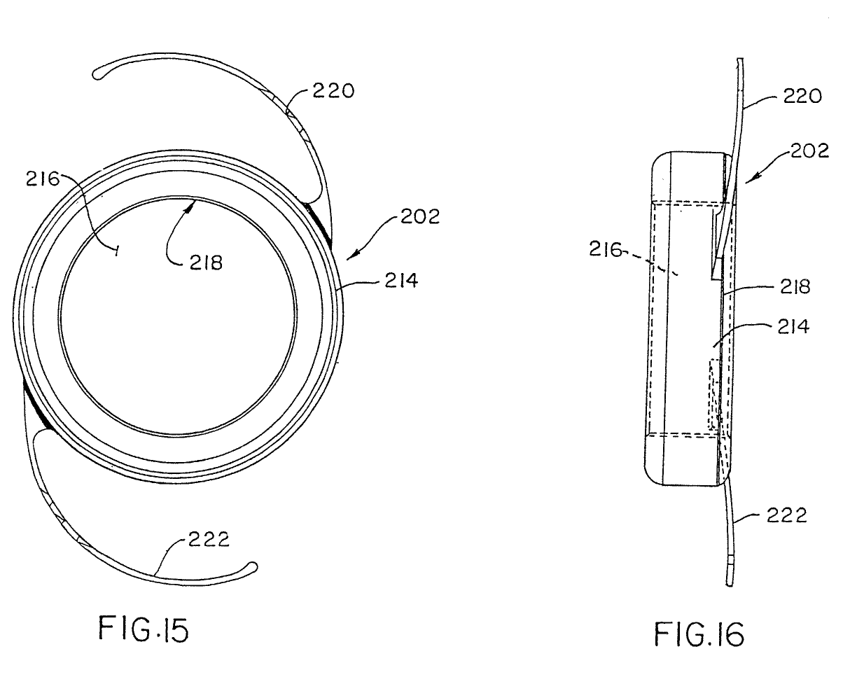

Another embodiment of the adjustable ocular implant is illustrated in FIGS. 13 through 16. The ocular implant includes an adjustable lens which is indicated generally by reference numeral 200 and a base annulus 202. Lens 200 includes a first or front plano-convex surface 204 and a second or back plano-convex surface 206. The front or first surface 204 includes at least one opening or hole 208 for the insertion of an adjustment tool. The two foregoing plano-convex surfaces are separated by an integral material thickness or body 210. The body has plurality of external threads 212 for the threaded insertion in the base annulus 202 as will be described below. It will be appreciated that the plurality of threads generally has a uniform radius.

However, it also will be noted that the extreme outer threads, 212A and 212B are comprised of the edge of the respective outer plano-convex surfaces and have a radius which is somewhat less than the radius of the middle threads. The reduced radii facilitate introduction and removal of the lens 200 into and out of the annulus 202.

The base annulus 202 is shown in greater detail in FIGS. 15 and 16 and includes a substantially circular body 214 having an opening 216 centrally therein. Opening 216 has at least one peripheral thread 218. The diameter of opening 216 is such so as to accommodate the introduction of the lens 200 by threadedly engaging the threaded surface 212 with thread 218. Furthermore, body 214 is of an appropriate depth or thickness so as to seat the lens 200 with the respective plano-convex surfaces extending beyond the plane of the body. Body 214 also includes a first arcuate haptic 220 and a second opposed arcuate haptic 222 to secure the insert in the capsular bag of the human eye after a cataract or, alternatively, secure the insert in the ciliary sulcus or anterior chamber.

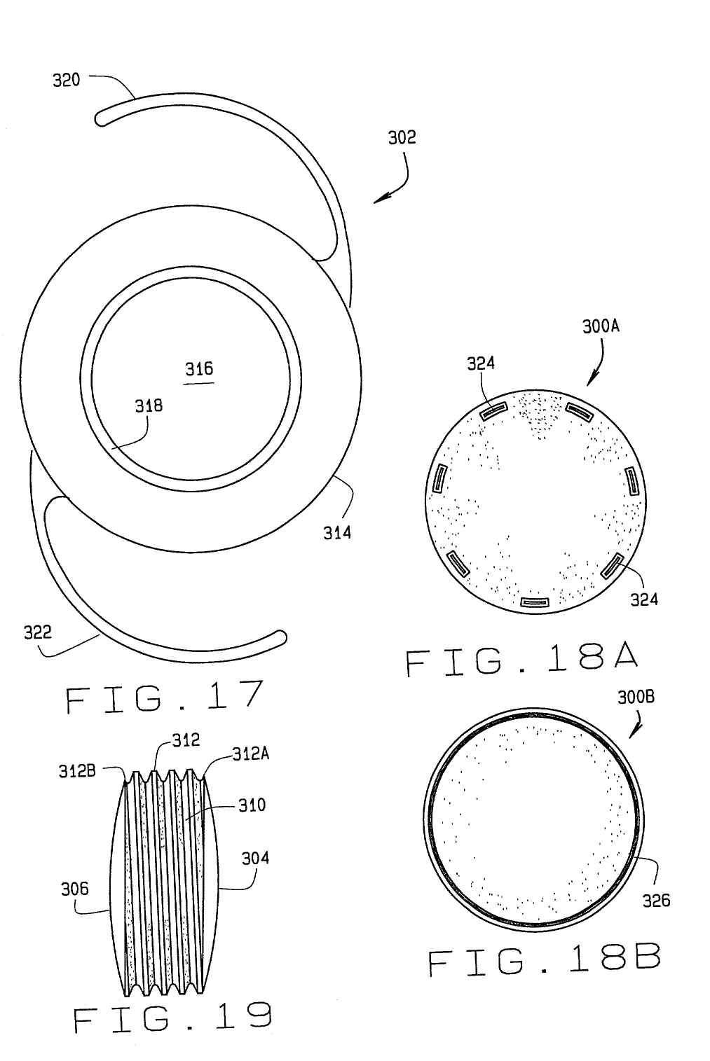

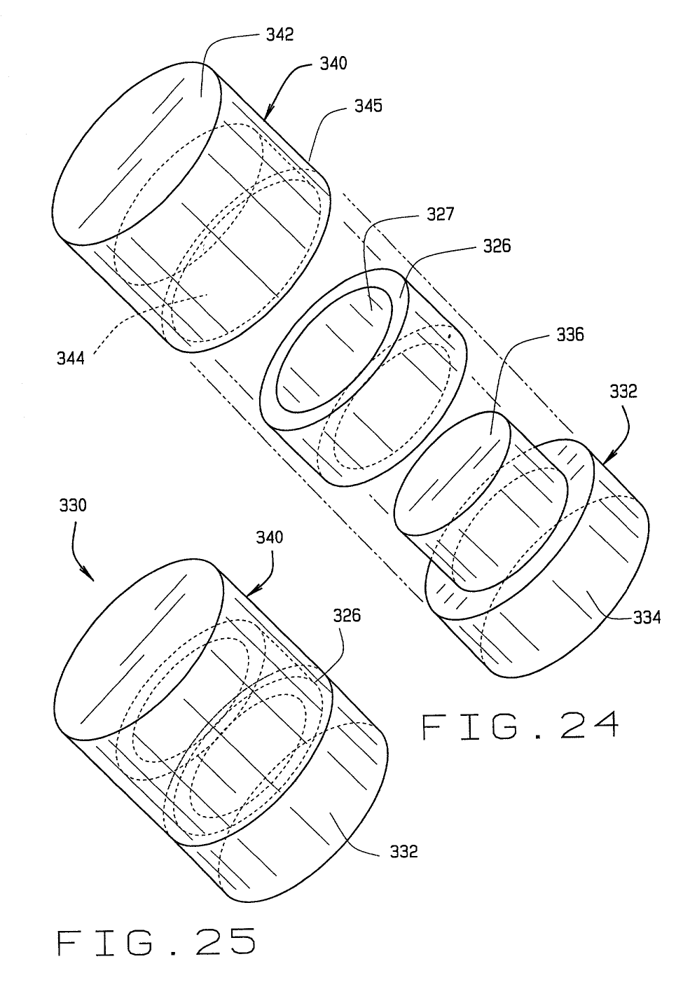

FIGS. 17 through 19B and 21 through 25 illustrate another embodiment of the adjustable ocular implant. The ocular implant includes an adjustable lens which is indicated generally by reference numeral 300A or 300B and a base annulus 302. Lens 300A or 300B includes a first or front plano-convex surface 304 and a second or back plano-convex surface 306. The two foregoing plano-convex surfaces are separated by an integral material thickness or body 310. The body has plurality of external threads 312 for the threaded insertion in the base annulus 302 as will be described below. It will be appreciated that each of the plurality of threads generally has a uniform radius. However, it also will be noted that the extreme outer threads, 312A and 312B are comprised of the edge of the respective outer plano-convex surfaces and have a radius which is somewhat less than the radius of the middle threads. The reduced radii facilitate introduction and removal of the lens 300A or 300B into and out of the annulus 202.

The base annulus 302 is shown in greater detail in FIG. 17 and includes a substantially circular body 314 having an opening 316 centrally therein. Opening 316 has at least one peripheral thread 318 The diameter of opening 216 is such so as to accommodate the introduction of the lens 300A or 300B by threadedly engaging the threaded surface 312 with thread 318. Body 314 also includes a first arcuate haptic 320 and a second opposed arcuate haptic 322 to secure the insert in the capsular bag of the human eye after a cataract or, alternatively, secure the insert in the ciliary sulcus or anterior chamber.

As can be seen, lens 300A includes a series of discrete magnetic sections 324 adjacent the periphery of the lens. The number of discrete magnetic sections may vary. The magnetic sections illustrated are equidistant apart. Lens 300B includes one continuous magnetic ring 326 around the peripheral edge of the lens.

The construction of a lens blank for forming a lens 300B is shown in detail in FIGS. 21-25. The assembled blank, indicated generally as reference numeral 330 in FIG. 25. The blank includes a magnet-mounting component 332, which can be referred to as a button, shown in FIG. 21. Button 332 has a front optical lens section 334 and a concentric stem 336. It will be noted that the front optical lens section 334 is relatively thick disc-shaped section. After the lens blank is assembled, lens section 334 can be appropriately machined to the desired optical curvature so as to meet the desired dioptric power. The blank includes magnetic ring 326 having an inner bore 327 of a diameter D that allows it to fit snugly and seat on stem 336. The magnetic ring 326 may additionally be fixed to the stem in any appropriate manner, such as an acceptable glue or the like so that it will not rotate on the stem when magnetic torque is applied as described below.

The blank also includes a cap 340 which has body 341 with first end 342 having an appropriate material thickness so that the end of the cap can be appropriately machined to the desired optical curvature to achieve the desired dioptric power after assembly. The opposite end of the cap has an inner bore 344. Bore 344 is dimensioned so that stem 336 with the magnetic ring mounted thereon can seat snugly in bore 344 and be secured in an appropriate manner such as gluing or the like.

Body 341 has an outer surface 345 that can be roll threaded to create external threads 310 (FIG. 19). Generally the cap and button are formed from PMMA. Once the various components are assembled, the button is pressed into the cap, securing the cap in a tight pressed fit between the stem and the cap. The assembled blank is subsequently roll-threaded and machined to the desired optical curvature.

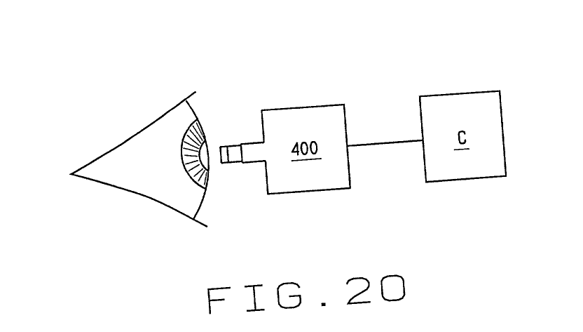

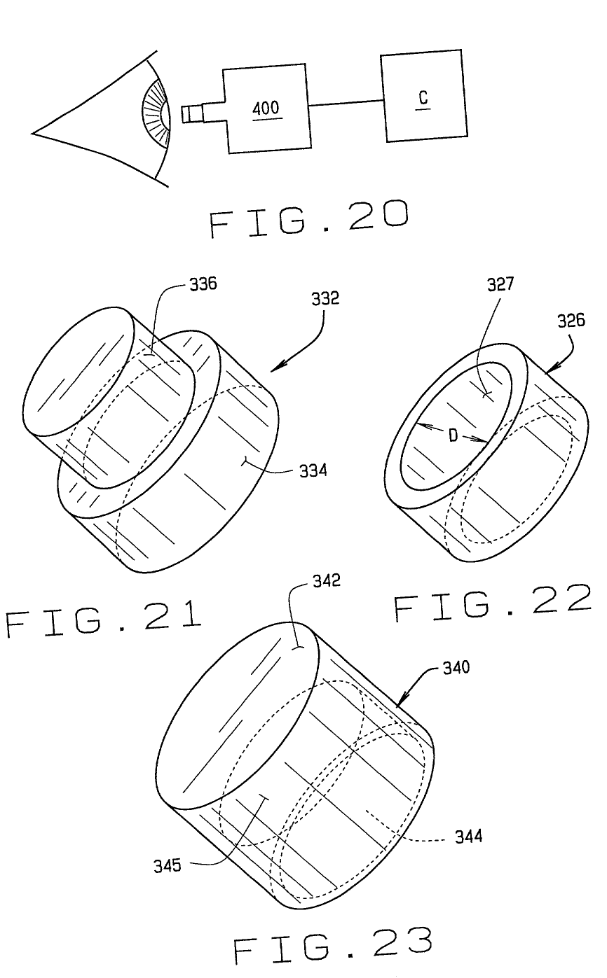

The magnetic sections 324 or ring 326 preferably is comprised of a rare earth magnetic material, more preferably a Samarian Cobalt magnetic material. In use, the intraocular device is positioned within the capsular bag, anterior chamber with haptics in the ciliary sulcus. The dioptric power of the lens can be adjusted by rotation of lens 300A or 300B within the annulus as described with regard to the other embodiments. However, lens 300A or 300B can be rotated by the extra ocular application of an appropriate metallic tool, such as a wand, knob, button or the like that is attracted to the magnetic sections (300A) or magnetic ring (300B). In a preferred embodiment, as shown in FIG. 20, the device is a iron filament electromagnetic device 400 as the external source to turn the lens. Such as device is positioned externally to the eye E which has the lens 300A or B implanted and can exactly control rotation of the lens. The electromagnetic device 400 can be operated by an appropriately programmed computer C for precise control. By manipulating the tool, the lens can be rotated within the annulus to the desired dioptric power. Any type of tool that will attract, or be appropriately attracted to, the magnet is contemplated by the invention.

In view of the various changes and modification just described, it will be appreciated that the foregoing description and accompanying drawings are intended to be illustrative only and should not be viewed in a limiting sense.

1. An adjustable intraocular insert comprising:
a base, said base comprising an annular body with an inner wall defining an opening, said body having at least one attachment means thereon, and
a lens mounted in the opening in the base, said lens having a first plano-convex surface and a second plano-convex surface with a material thickness between said first plano-convex surface and second plano-convex surface, said material thickness including a threaded surface for the threaded engagement of the base, said threaded surface include a lead thread having a radius less than a radius of said threaded surface to facilitate introduction of the lens into the base, whereby rotation of the lens within the base will affect a change in focusing power of the lens, said lens having means thereon to facilitate rotation of the lens within the base.

2. The adjustable intraocular insert of claim 1 wherein the means thereon for facilitating rotation of the lens include structures on the lens for insertion of a tool to facilitate rotation of the lens.

3. The adjustable intraocular insert of claim 1 wherein the means thereon for facilitating rotation of the lens include magnetic material.

4. An in situ adjustable intraocular insert functioning as an artificial lens for insertion into the lens capsule of the eye comprising:
a base, said base comprising a compressible and resilient outer annular body designed to fit within the lens capsule of the eye, said base having an inner wall defining an opening, the inner wall of said base comprising a threaded inner wall defining a circular opening;
a lens mounted in the circular opening in the base, and said lens threadedly engaging to the threaded inner wall of the base;
said lens having a first plano-convex surface and a second plano-convex surface with a material thickness between said first plano-convex surface and said second plano-convex surface, the combined shape of said lens mounted in said base having a shape complimentary to the natural lens of the eye and designed to fit within the lens capsule;
the material thickness of the lens being sufficient to include a threaded surface for the threaded engagement within the threaded opening of the base, said threaded surface including a lead thread having a radius less than a radius of said threaded surface to facilitate introduction of the lens into the base, whereby the in situ rotation of the lens within the effect a change in focusing of the power of the lens after implantation.

5. The intraocular insert of claim 4 wherein said lens is removable from said base so as to allow the replacement of said lens.

6. The intraocular insert of claim 4 wherein said base includes at least one curvilinear haptic on an outer edge thereof for securing the base annulus within the lens capsule of the eye.

7. The intraocular insert of claim 4 wherein said annular base has at least one of opening formed therein for the introduction of a surgical instrument, said openings also functioning as indicia of dioptric power.

8. The intraocular insert of claim 5 wherein said lens has at least one opening formed therein for the introduction of a surgical instrument, and said opening also functioning as an indicia of dioptric power.

9. An adjustable intraocular insert functioning as an artificial lens for insertion into one of the lens capsule, ciliary sulcus, or anterior chamber of the eye, comprising:
a base, said base comprising a compressible and resilient outer annular body designed to fit within the lens capsule or other portions of the eye, said base having an inner wall defining an opening, the inner wall of said base comprising an threaded inner wall defining a circular opening;
a lens mounted in the circular opening of the base, and said lens threadedly engaging to the inner threaded wall of said base;
said lens having at least one convex surface, and a second surface, with a material thickness between said convex surface and said other surface, the combined shape of the lens mounted in said base having a shape complementary to the natural lens of the eye and designed to fit within at least the lens capsule;
the material thickness of the lens being sufficient to include a threaded surface for its threaded engagement within the threaded opening of the base,
said lens having a magnetic material embedded therein and arranged approximate the outer periphery of the lens, whereby the in situ rotation of the lens through the use of magnetic force effecting a fine adjustment in the threaded engagement between the lens and its base and furnishing focusing of the power of the lens after implantation through the exertion of magnetic force exteriorly of the eye without necessitating any further ophthalmic surgical operation.

10. The adjustable intraocular insert of claim 9 wherein said base further comprises haptic attachment means provided thereon and extending laterally therof,

11. The intraocular insert of the claim 9 wherein said magnet comprises an annular ring.

12. The intraocular insert of claim 9 wherein said magnet comprises equally spaced segments of magnetic material.

13. The intraocular insert of claim 9 wherein said lens is bi-convex.

14. The intraocular insert of claim 9 wherein said lens has a convex surface on one surface, and is planar on the opposite surface.

15. The intraocular insert of claim 9 wherein said magnetic material is a rare earth magnetic material.

16. The intraocular insert of claim 15 where said rare earth magnetic material is Samarian Cobalt.

17. The intraocular insert of claim 9 wherein said threaded surface of the lens include a lead thread having a radius less than a radius of said threaded surface to facilitate the introduction of the lens into the base.

18. An in situ adjustable intraocular insert functioning as an artificial lens for insertion into the eye comprising:
a base, said base comprising a compressible and resilient outer annular body having at least one haptic thereon, said base having an inner wall defining an opening, the inner wall of said base comprising a threaded inner wall defining a circular opening;
a lens mounted in the circular opening in the base, said lens having a first plano-convex surface, with means thereon for effecting rotational movement of the lens in the circular opening of the base, and a second plano-convex surface with a material thickness between said first plano-convex surface and said second plano-convex surface, the material thickness of the lens being sufficient to include a threaded surface for the threaded engagement within the threaded opening of the base, said threaded surface including a lead thread having a radius less than a radius of said threaded surface to facilitate introduction of the lens into the base, whereby the in situ rotation of the lens within the effect a change in focusing of the power of the lens after implantation.

19. An in situ adjustable intraocular insert functioning as an artificial lens for insertion into the eye comprising:
a base, said base comprising an outer annular body and having an inner wall defining an opening, the inner wall of said base comprising a threaded inner wall defining a circular opening;
a lens assembly mounted in the circular opening in the base, said lens assembly including a button component with a lens section having a desired optical curvature on one side and a concentric magnet mounting section formed thereon, a magnetic ring positioned around said magnet mounting section, and a cap positioned over said magnetic ring and stem, said cap including a lens section having a desired optical curvature and an outer surface for threadedly engaging the inner wall of the annular body.

20. The in situ adjustable intraocular insert of claim 16 wherein said base, button component and cap are formed from PMMA.

21. The in situ adjustable intraocular insert of claim 18 wherein said magnetic ring is formed from samarium cobalt

22. A blank for forming a lens included in a lens assembly mounted in the circular opening in the base, said blank including a button component with a lens section capable of being formed into a desired optical curvature and a concentric magnet mounting section formed thereon, a magnetic ring positioned around said magnet mounting section, and a cap positioned over said magnetic ring and stem, said cap including a lens section capable of being formed into a desired optical curvature and a body portion having a material thickness capable of having threads formed thereon.
